(19) United States
(12) Patent Application Publication
(10) Pub. No.: US 2025/0140397 A1
(43) Pub. Date: May 1, 2025

(54) ELECTRONIC DEVICE PROVIDING SLEEP STATUS DETERMINATION USING ARTIFICIAL INTELLIGENCE, OPERATION METHOD OF THE SAME AND SYSTEM

(71) Applicants: CODEVISION INC., Seoul (KR); RESEARCH & BUSINESS FOUNDATION SUNGKYUNKWAN UNIVERSITY, Suwon-si (KR)

(72) Inventors: Eung Yeol SONG, Seoul (KR); Kiyong KIM, Seoul (KR); Do-Hyung KIM, Changwon-si (KR); Byeonggu JEON, Changwon-si (KR)

(73) Assignees: CODEVISION INC., Seoul (KR); RESEARCH & BUSINESS FOUNDATION SUNGKYUNKWAN UNIVERSITY, Suwon-si (KR)

(21) Appl. No.: 18/932,651

(22) Filed: Oct. 31, 2024

(30) Foreign Application Priority Data
Oct. 31, 2023 (KR) 10-2023-0148659

(51) Int. Cl. G16H 40/63

(52) U.S. Cl. CPC G16H 40/63

(57) ABSTRACT

According to various embodiments, the electronic device includes a communication device, a storage device storing a first artificial intelligence model trained for sleep state determination, and at least one processor, wherein the processor acquires first initial biometric information regarding oxygen saturation and second initial biometric information regarding ECG data of a sleeping user via a sensor device, the processor converts this initial information into multidimensional first and second biometric information, identifies ECG peak points and intervals, and inputs these data into the AI model to determine the user's sleep state, and the sleep state information is then transmitted to a display device connected to the electronic device for output.

### CROSS-REFERENCE TO RELATED APPLICATIONS

This application claims priority under 35 U.S.C. § 119 to Korean Patent Application No. 10-2023-0148659 filed on Oct. 31, 2023, which are incorporated by reference herein in their entirety.

### BACKGROUND

### 1. Field

Various embodiments disclosed in this document relate to an electronic device providing sleep status determination using artificial intelligence, its operation method, and a related system.

### 2. Description of Related Art

As the interest in health has increased, a device for determining health conditions by measuring and analyzing various bio-signals has been developed. In particular, sleep occupies about 30% or more of the day, and it is more important than anything that it manages sleep well in health management.

Meanwhile, as such sleep is recognized as an important factor that affects physical or mental health, interest in maturity has increased, and therefore, interest in determining sleep conditions has increased.

A test for determining such sleep conditions includes a sleep multifunction test. The sleep multifunction test is a test that measures the quality and amount of sleep and finds sleep diseases and sleep-related disorders. In general, the sleep multifunction test measures the physiological and physical signals from the human body during sleep and finds various sleep diseases and sleep disorders.

Sleep diseases may include diseases such as insomnia, hypersomnia, sleep apnea syndrome, night scene, monuments, lemm sleep disorder, unconscious lemm sleep abnormalities, and sleep epilepsy.

In the related art, in order to determine a sleep state, sensor equipment measuring physiological and physical signals have been used to determine a sleep state of a user of a person. However, in the related art, in order to determine a sleep state and determine a sleep disease, a bio-signal obtained through the sensor equipment has to be analyzed by a professional and manually determined. Therefore, there is a problem in that it takes considerable time and cost to determine a sleep disease and accuracy is lowered.

In addition, when a device for automatically determining a sleep disease is used, there is a problem in that the accuracy of determining a sleep state is lowered. For example, the related art sleep state determining apparatus determines a sleep state by using biometric information obtained from a user in sleep as it is. In this case, the biometric information is information obtained for a sleep time (e.g., about 7 hours) of the user and has a large amount, and the sleep state determined by using the large amount of biometric information has a problem in that it has a lower accuracy due to noise data, a considerable time to determine, and a storage space in the apparatus cannot be efficiently used.

### SUMMARY

According to various embodiments, the electronic device comprises a communication device, a storage device storing a first artificial intelligence model trained to determine a sleep state, and at least one processor. The at least one processor is configured to obtain first initial biometric information regarding oxygen saturation of a sleeping user and second initial biometric information regarding electrocardiogram (ECG) data through at least one sensor device. The at least one processor obtains first biometric information by converting the first initial biometric information into a multidimensional form, and it obtains a plurality of peak points of the ECG data and time intervals between the plurality of peak points based on the second initial biometric information. The processor further obtains second biometric information based on the plurality of peak points and the time intervals between the plurality of peak points and inputs the first biometric information and the second biometric information into the first artificial intelligence model to obtain sleep state information of the user. The communication device may be configured to transmit the sleep state information to a display device connected to the electronic device for output.

According to various embodiments, the method of operation of the electronic device comprises obtaining first initial biometric information regarding oxygen saturation and second initial biometric information regarding ECG data of a sleeping user through at least one sensor device; obtaining first biometric information by converting the first initial biometric information into a multidimensional form; obtaining a plurality of peak points of the ECG data and time intervals between the plurality of peak points based on the second initial biometric information; obtaining second biometric information based on the plurality of peak points and the time intervals between the plurality of peak points; inputting the first biometric information and the second biometric information into the first artificial intelligence model to obtain the user's sleep state information, wherein the first artificial intelligence model is trained to determine the sleep state; and transmitting the sleep state information to a display device connected to the electronic device for output.

According to various embodiments, the health condition diagnosis system comprises at least one sensor device configured to obtain biometric information including oxygen saturation and ECG data of a user over a period of time, a display device, and a server storing a first artificial intelligence model trained to determine a sleep state. The server is configured to obtain, via the at least one sensor device, first initial biometric information regarding the user's oxygen saturation and second initial biometric information regarding the user's ECG data; obtain first biometric information by converting the first initial biometric information into a multidimensional form; obtain a plurality of peak points of the ECG data and time intervals between the plurality of peak points based on the second initial biometric information; obtain second biometric information based on the plurality of peak points and the time intervals between the plurality of peak points; input the first biometric information and the second biometric information into the first artificial intelligence model to obtain health condition information of the user, and transmit the health condition information to the display device, wherein the display device is configured to display a screen including the health condition information.

The electronic device according to various embodiments of this disclosure can diagnose the user's health condition based on biometric information obtained through at least one sensor.

For example, the electronic device according to various embodiments can diagnose the user's health condition by determining the user's sleep state based on the user's biometric information.

According to various embodiments, the electronic device can accurately determine or assess the user's sleep state using an artificial intelligence model trained to determine the user's sleep state based on the user's biometric information.

According to various embodiments, the electronic device can automatically diagnose the user's sleep state using an artificial intelligence model, thereby reducing the cost and time required for sleep state diagnosis.

According to various embodiments, the electronic device can improve the accuracy of sleep state determination, reduce the time required for assessment, and efficiently use storage by converting and utilizing the biometric information of the sleeping user.

### BRIEF DESCRIPTION OF THE DRAWINGS

FIG. 1 is a block diagram of a health diagnosis system for diagnosing a health condition of a user.

FIG. 2 is a block diagram of an electronic device for determining a sleep state of a user.

FIG. 3 illustrates a concept of controlling a function related to a sleep state determination of an electronic device.

FIG. 4 is a flowchart illustrating a method of determining a sleep state by an electronic device.

FIG. 5 is a flowchart illustrating a method of obtaining second biometric information by an electronic device.

FIG. 6 is a diagram for explaining obtaining first biometric information based on first initial biometric information.

FIG. 7A is a diagram for explaining obtaining second biometric information based on second initial biometric information.

FIG. 7B is a diagram for explaining obtaining second biometric information based on second initial biometric information.

FIG. 8 is a flowchart illustrating an operation of training a first AI model by an electronic device.

FIG. 9 is a diagram for explaining obtaining label data by an electronic device.

FIG. 10 is a diagram for explaining obtaining sleep state information using a first AI model.

FIG. 11 is a flowchart illustrating a method of obtaining a second AI model based on a re-training request.

FIG. 12 is a flowchart illustrating operations of devices in a system for determining a sleep state.

FIG. 13 illustrates a user interface output through a display device.

In relation to the description of the drawings, the same or similar reference numerals may be used for the same or similar components.

### DETAILED DESCRIPTION

Specific structural or functional descriptions of various embodiments are merely illustrated for the purpose of describing the various embodiments, and they should not be construed as being limited to the embodiments described in this specification or the application.

Various embodiments can be variously modified and have various forms, and thus various embodiments are illustrated in the drawings and will be described in detail in this specification or the application. However, it should be understood that the matter disclosed from the drawings is not intended to specify or limit various embodiments, but includes all modifications, equivalents, and alternatives included in the spirit and scope of the various embodiments.

The terms first and/or second, etc., may be used to describe various components, but the components should not be limited by the terms. The terms are only for the purpose of distinguishing one component from another component, for example, the first component may be named a second component, and similarly, the second component may be named a first component, without deviating from the scope of rights according to the concept of the present disclosure.

When an element is referred to as being “connected” or “connected” to another component, it should be understood that the element may be directly connected or connected to the other component, but other components may be present in the middle. On the other hand, when an element is referred to as being “directly connected” or “directly connected” to another component, it should be understood that there is no intervening component. Other expressions that describe the relationship between components, that is, “between” and “immediately between” or “adjacent to” and “directly adjacent to” should be interpreted as well.

The terminology used in this specification is used merely to describe a specific embodiment, and is not intended to limit various embodiments. The singular expression includes plural expressions unless the context clearly dictates otherwise. In this specification, it should be understood that the terms “include” or “have” are intended to designate the presence of stated features, numbers, steps, operations, components, parts or combinations thereof, but do not preclude the presence or addition of one or more other features, numbers, steps, operations, components, parts or combinations thereof.

Unless defined otherwise, all terms used herein, including technical or scientific terms, are the same meaning as commonly understood by one of ordinary skill in the art to which this disclosure belongs. Terms such as those defined in commonly used dictionaries should be interpreted to have a meaning that is consistent with the contextual meaning in the relevant art, and is not interpreted in an ideal or overly formal sense unless clearly defined in this specification.

Hereinafter, the present disclosure will be described in detail with reference to the preferred embodiments of the present disclosure with reference to the accompanying drawings. The same reference numerals presented in each figure indicate the same elements.

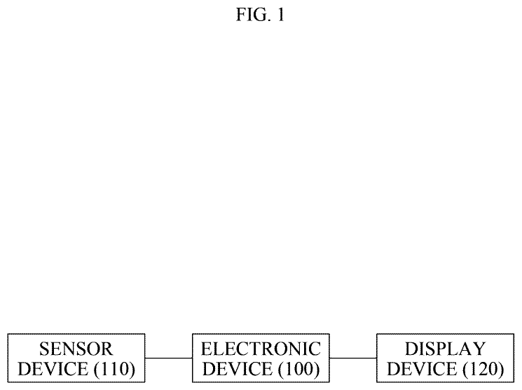

FIG. 1 is a block diagram of a health diagnosis system for diagnosing a health condition of a user.

Referring to FIG. 1, a health diagnosis system for diagnosing a user's health condition is illustrated. According to various embodiments, the health diagnosis system may include an electronic device 100, at least one sensor device 110, and a display device 120. However, the health diagnosis system may be implemented to include more devices and/or less devices, without being limited to the described and/or illustrated examples. In addition, at least two or more of the devices illustrated in the health diagnosis system may be implemented as one device.

According to various embodiments, the electronic device 100, the sensor device 110, and the display device 120 may be connected by electrical, mechanical, or wired or wireless communication. According to various embodiments, the electronic device 100, the sensor device 110, and the display device 120 may be indirectly connected. For example, the electronic device 100 may obtain biometric information obtained through the sensor device 110 through a data interworking device (e.g., a data interworking module, a hub device).

According to various embodiments, the electronic device 100 may diagnose the health state of the user based on the bio signal of the user (e.g., the subject) obtained through the sensor device 120. For example, the electronic device 100 may determine the sleep state of the user based on information about the oxygen saturation of the user and information about the electrocardiogram obtained through the sensor device 120.

According to various embodiments, the electronic device 100 may use an artificial intelligence model in determining the sleep state of the user. For example, the electronic device 100 may obtain an artificial intelligence model trained to determine a sleep state based on biometric information of the user and determine the sleep state of the user using the artificial intelligence model. For example, the electronic device 100 may process information about the oxygen saturation of the user and information about the electrocardiogram, and input the information about the oxygen saturation and the information about the electrocardiogram to the artificial intelligence model to obtain information about the sleep state of the user.

According to various embodiments, the electronic device 100 may obtain a request for training the artificial intelligence model, a request for retraining the artificial intelligence model, and/or a request for determining the sleep state of the user using the artificial intelligence model. In various embodiments, the electronic device 100 may provide a function corresponding to the request based on obtaining the requests. The requests may be obtained through an input device (e.g., a touch panel of the display device 120) connected to the electronic device 100. Therefore, the health diagnosis system may further include an input device or an output device, without being limited to the devices illustrated in FIG. 1.

According to various embodiments, the electronic device 100 may provide the user's sleep state information through the display device 120. For example, the electronic device 100 may transmit the sleep state information to the display device 120 so that the display device 120 displays a screen including sleep state information. According to various embodiments, information obtained by various functions provided by the electronic device 100 may be provided to the user through the display device 120. However, the information obtained by various functions provided by the electronic device 100 may be provided to a user through another output device (e.g., a speaker) without being limited to the above example. To this end, the electronic device 100 may be connected to various devices without being limited to the type of the device.

According to various embodiments, the sensor device 110 may represent a sensor configured to obtain a biometric signal of a user. The sensor device 110 is expressed as a single device for convenience of explanation, but may be configured with a plurality of sensor devices 110, and may be configured with one sensor device 110 including a plurality of sensors. For example, the sensor device 110 may include a sensor that may obtain information about the ECG of the user and a sensor that may obtain information about the oxygen saturation of the user. For example, the sensor device 100 may include at least one of a photoplethysmogram (PPG) sensor, an electrocardiogram (ECG) sensor, a bioimpedance (BIA) sensor, an optical fiber oxygen sensor, a membrane oxygen sensor, and an ultrasonic sensor.

According to various embodiments, the sensor device 120 may be provided (e.g., contacted, worn, attached, or the like) to at least a part of the body of the user who is sleeping, and obtain biometric information while the user is in the sleep state. According to an embodiment, the sensor device 110 may obtain sensor information related to at least one of the heart rate, blood pressure, blood sugar, blood volume, or oxygen saturation of the user through the PPG sensor. For example, when a user wears the sensor device 110 or takes a sleep while the sensor device 110 is in contact with a part of the user's body, the PPG sensor facing the user's skin may irradiate light through a light emitting unit, and the irradiated light reflected from the user's skin or transmitted through the skin may be detected through a light receiving unit to convert the detected light into an electric signal, and the converted electric signal may be processed to measure at least one of the user's heart rate, blood pressure, blood sugar, blood volume, or oxygen saturation. According to an embodiment, the sensor device 110 may obtain sensor information related to the ECG of the user through the ECG sensor. For example, when a user wears the sensor device 110 or takes a sleep while the sensor device 110 is in contact with a part of the user's body, the ECG of the user may be measured by measuring the change in potential according to the activity of myocardial cells through the electrode of the ECG sensor facing the user's skin.

According to various embodiments, the sensor device 120 may transmit the obtained biometric information to the electronic device 100. For example, the sensor device 120 may establish a direct (e.g., wired) communication channel or a wireless communication channel to transfer the obtained biometric information to the electronic device 100.

According to various embodiments, the sensor device 120 may store biometric information of at least one user. For example, the sensor device 120 may accumulate and store biometric information obtained by being provided to at least a part of the user's body. Therefore, the sensor device 120 sequentially provided to a plurality of users may store biometric information about a plurality of users. In this case, the sensor device 120 may identify each of the plurality of users and store biometric information.

According to various embodiments, the display device 120 may visually provide information to the outside (e.g., the user). For example, the display device 120 may display various contents (e.g., text, image, video, icon, and/or symbols). According to various embodiments, the display device 120 may include a display. For example, the display device 120 may include a liquid crystal display (LCD), a light-emitting diode (LED) display, or an organic light-emitting diode (OLD) display.

According to various embodiments, the display device 120 may display various information obtained from the electronic device 100. For example, the display device 120 may obtain the sleep state information of the user from the electronic device 100 and output a visual object (e.g., a screen for displaying the sleep state information) including the sleep state information.

According to various embodiments, the display device 120 may include a touch sensor configured to sense a touch or a pressure sensor configured to measure the intensity of force generated by the touch. According to various embodiments, the display device 120 may display an execution screen and obtain a user touch input on the execution screen using at least one of the touch sensor or the pressure sensor. For example, the display device 120 may display an execution screen guiding a training request for artificial intelligence models for analyzing sleep states, a re-training request for the artificial intelligence models, and/or a user's sleep state determination request using the artificial intelligence models, and obtain a user input on the screen. In various embodiments, the display device 120 may transfer the user input to the electronic device 100.

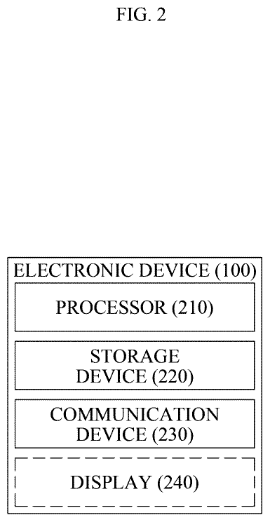

According to various embodiments, the electronic device 100 and the display device 120 may be implemented as a single device. In this case, the electronic device 100 may control the display device 120 through the processor to display the execution screen. An example of the case where the display device 120 is included in the electronic device 100 will be described below with reference to FIG. 2.

According to various embodiments, the display device 120 may refer to various devices including a display. For example, it may include a portable communication device (e.g., a smartphone), a computer device, a portable multimedia device, a portable medical device, a camera, a wearable device, or a home appliance. The display device 120 according to an embodiment of the present disclosure is not limited to the above-described devices.

According to various embodiments, the electronic device 100, the sensor device 110, and/or the display device 120 may include an interface (not shown). In an embodiment, the interface may support one or more specified protocols that the electronic device 100, the sensor device 110, and/or the display device 120 may be used to connect with an external electronic device (e.g., the electronic device 100, the sensor device 110, and/or the display device 120) directly or wirelessly. According to an embodiment, the interface may include, for example, a high definition multimedia interface (HDMI), a universal serial bus (USB) interface, an SD card interface, or an audio interface.

FIG. 2 is a block diagram of an electronic device that determines the sleep state of a user.

Referring to FIG. 2, the electronic device 100 may include a processor (PROCESSOR) 210, a storage device 220, a communication device 230 and/or a display 240 (e.g., the display device 120 of FIG. 1). The above-listed components may be operatively or electrically connected to each other. some of the components of the electronic device 100 illustrated in FIG. 2 may be modified, deleted, or added.

According to various embodiments, the electronic device 100 may include a processor 210. The processor 210 may include hardware for executing a command, such as a command constituting a computer program. For example, the processor 210 may search for (or patch) the command from the internal register, the internal cache, the storage device 220 (including the memory), decode and execute the command, and store the result in the internal register, the internal cache, and the storage device 220 to execute the command.

In various embodiments, the processor 210 may execute software (e.g., a computer program) to control at least one other component (e.g., hardware or software component) of the electronic device 100 connected to the processor 210, and may perform various data processing or calculations. According to various embodiments, as at least a part of the data processing or calculation, the processor 210 may store the command or data received from the other component (e.g., the communication device 230) in volatile memory, process the command or data stored in the volatile memory, and store the result data in non-volatile memory.

According to various embodiments, the processor 210 may include at least one of a central processing unit (CPU), a graphics processing unit (GPU), a micro controller unit (MCU), a sensor hub, an auxiliary processor, a communication processor, an application processor, an application specific integrated circuit (ASIC), a field programmable gate array (FPGA), or a neural processing unit (NPU), and may have a plurality of cores.

According to various embodiments, the processor 210 (e.g., the neural network processing device) may include a hardware structure specialized for processing an artificial intelligence model. The artificial intelligence model may be generated through machine training. The training algorithm may include, for example, supervised training, unsupervised training, semi-supervised training, or reinforcement training, but is not limited to the above example. The artificial intelligence model may include a plurality of artificial neural network layers. The artificial neural network may be one of a deep neural network (DNN), a convolutional neural network (CNN), a recurrent neural network (RNN), a restricted Boltzmann machine (RBM), a deep belief network (DBN), a bidirectional recurrent deep neural network (BRDNN), a deep Q-network, or a combination of two or more of the above, but is not limited to the above example. The artificial intelligence model may additionally or alternatively include a software structure in addition to the hardware structure.

According to various embodiments, the processor 210 may obtain various biometric information of the user through at least one sensor device (e.g., the sensor device 110 of FIG. 1). For example, the processor 210 may obtain first initial biometric information about the oxygen saturation of the user under sleep and second initial biometric information about the ECG obtained through the at least one sensor device.

According to various embodiments, the first initial biometric information about the oxygen saturation may mean information about the oxygen saturation obtained by providing the at least one sensor device 110 to a part of the user under sleep. The oxygen saturation is the ratio of oxygen saturated hemoglobin to total hemoglobin in blood, and the first initial biometric information may include a 1-dimensional signal graph obtained through sensors for measuring oxygen saturation. For example, the first initial biometric information may include information indicating the magnitude of oxygen saturation per hour.

Meanwhile, the normal arterial blood oxygen saturation level of humans is 95-100%, which is considered low if the value is less than 90%, and is classified as hypoxemia, and the arterial blood oxygen level of less than 80% may damage organ functions such as brain and heart, and the continuously low oxygen level may lead to respiratory or cardiac paralysis. The oxygen saturation level is determined by measuring the ratio of the hemoglobin-binding site occupied by oxygen in the blood stream, and most hemoglobin is deoxygenated at low oxygen partial pressure, and at about 90%, the oxygen saturation level increases according to the oxygen-hemoglobin dissociation curve. The pulse oxygen meter represents the oxygen saturation level depending on the light absorption characteristic of the saturated hemoglobin. Therefore, based on the oxygen saturation level of the sleep user, the sleep state, such as sleep apnea, which is a disease in which the user does not temporarily breathe during sleep, may be determined.

According to various embodiments, the second initial biometric information about the electrocardiogram may refer to information about the oxygen saturation level obtained by providing at least one sensor device 110 to a part of the body of the sleep user. The electrocardiogram is a record of the contraction and relaxation stages of the heart, and may include a graph of electrical activities of the atrium and the ventricle. Therefore, the second initial biometric information may include information indicating the size of the heartbeat per hour of the sleep user.

Meanwhile, the breathing of the user and the electrocardiogram are closely related. For example, when the breathing of the user is temporarily interrupted or shallowed, the supply of oxygen may decrease, and accordingly, a load may be applied to the heart. Therefore, based on the electrocardiogram pattern of the sleep user, the sleep state, including sleep apnea, which is a disease in which the user does not temporarily breathe during sleep, may be determined.

According to various embodiments, the processor 210 may determine the sleep state of the user by using the first initial biometric information about the oxygen saturation level and the second initial biometric information about the electrocardiogram. In this case, the processor 210 may determine the sleep state of the user through the first biometric information and the second biometric information obtained by converting the first initial biometric information and the second initial biometric information.

Specifically, according to various embodiments, the processor 210 may obtain the first biometric information obtained by converting the first initial biometric information into multidimensional first biometric information. For example, the processor 210 may obtain the first biometric information obtained by converting the first initial biometric information into two-dimensional first biometric information (e.g., two-dimensional graph). In one embodiment, the processor 210 may convert the first initial biometric information into first biometric information by performing one of various conversion methods. For example, the processor 210 may obtain the first biometric information by converting the first initial biometric information through at least one of Fourier transform, Wavelet transform, Wavelet spectrogram, and Mel-Frequency Cepstral Coefficients (MFCC). According to various embodiments, the processor 210 may obtain the first biometric information by converting the first initial biometric information into multidimensional first biometric information (e.g., two-dimensional graph).

According to various embodiments, the processor 210 may process the second initial biometric information to obtain second biometric information converted into a multidimensional form. For example, the processor 210 may obtain second biometric information by converting the second initial biometric information including the electrocardiogram that represents the heartbeat by time into two-dimensional dimensions.

According to various embodiments, the processor 210 may obtain second biometric information by converting second initial biometric information into two dimensions. In this case, the processor 210 may process the second initial biometric information and convert it into two-dimensions. For example, the processor 210 may obtain a plurality of peak points of the electrocardiogram and a time interval between the plurality of peak points based on the second initial biometric information. Then, the processor 210 may obtain second biometric information based on the plurality of peak points and a time interval between the plurality of peak points.

According to various embodiments, the processor 210 may obtain a tachogram of a heart rate variance related to the electrocardiogram based on the time interval between the plurality of peak points and the plurality of peak points, and may convert the tachogram of the heart rate variance into a multidimensional image to obtain the second biometric information. The processor 210 converting the tachogram of the heart rate variability into a multidimensional may be obtained by performing an operation similar to that of obtaining the first biometric information.

According to various embodiments, the processor 210 converts and uses the second initial biometric information into multi-dimensional second biometric information without utilizing the second initial biometric information as it is, thereby clearly visualizing the characteristics of the data to increase the accuracy of determining the sleep state. In addition, the processor 210 does not convert the electrocardiogram into a multidimensional basis, extracts a plurality of peak points and intervals between peak points, obtains a tachogram of a heart rate variability, and converts the tachogram of the heart rate variability into a multidimensional form basis to obtain second biometric information, thereby removing noise in the second initial biometric information, and reducing the size of the second initial biometric information. Therefore, it is possible to improve the quality degradation of the sleep state determination due to noise and reduce the size of the second initial biometric information to efficiently use the storage device 220.

According to various embodiments, the processor 210 may obtain sleep state information by inputting the first biometric information and the second biometric information into a first artificial intelligence model that is trained to determine the sleep state. For example, the processor 210 may store a first artificial intelligence model trained to determine a sleep state in the storage device 220, and obtain sleep state information output by inputting the first biometric information and the second biometric information to the first artificial intelligence model.

According to various embodiments, the sleep state information may indicate information related to the user's sleep disease. For example, the processor 210 may determine the sleep condition of the user by determining whether the disease related to sleep disorders such as sleep apnea syndrome, obstructive sleep apnea syndrome, sleep apnea, and snoring, which are sleep diseases of the user using the first artificial intelligence model. Therefore, the sleep state information may include whether the user has a sleep disease.

According to various embodiments, the electronic device 100 may include a storage device 220. According to various embodiments, the storage device 220 may include a mass storage for data or commands. For example, the storage device 220 may include a hard disk drive (HDD), a floppy disk drive, a flash memory, an optical disk, a magneto-optical disk, a magnetic tape, or a universal serial bus (USB) drive or a combination of two or more thereof. In various embodiments, the storage device 220 may include non-volatile, solid-state memory, and read-only memory (rom). These roms may be mask-programmed roms, programmable roms (PROM), erasable proms (EPROM), electrically erasable proms (EEPROM), electrically alterable roms (EAROM), or flash memories or a combination of two or more thereof.

Although this disclosure describes and illustrates a specific storage device, this disclosure contemplates any suitable storage device, and according to various embodiments, the storage device 220 may be inside or outside the electronic device 100.

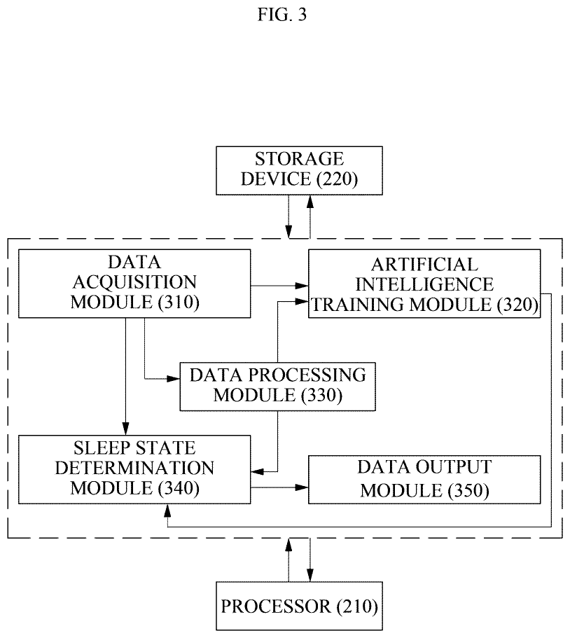

According to various embodiments, the processor 210 may store a module providing a function related to the determination of the sleep state described with reference to FIG. 3 in the storage device 220.

According to various embodiments, the processor 210 may execute calculations or data processing related to control and/or communication of at least one other components of the electronic device 200 using instructions stored in the storage device 220. According to various embodiments, the storage device 220 may store various data used by at least one component (e.g., the processor 210) of the electronic device 200. The data may include, for example, software (e.g., a program) and input data or output data for commands related thereto.

According to various embodiments, the program may be stored in the storage device 220 as software, and may include, for example, an operating system, middleware, or an application. According to various embodiments, the storage device 220 may store instructions that cause the processor 210 to process data or control components of the electronic device 200 to perform the operations of the electronic device 200 when executed. The instructions may include code generated by a compiler or code that can be executed by an interpreter.

According to various embodiments, the storage device 220 may store various information obtained through the processor 210. For example, the storage device 220 may store biometric information of a user obtained from at least one sensor device 110. For example, the storage device 220 may store a set of biometric information of at least one user obtained through the processor 210. In addition, the storage device 220 may store various artificial intelligence models. For example, the storage device 220 may store an artificial intelligence model learned and generated by the processor 210. Therefore, the storage device 220 may store the first artificial intelligence model trained by the processor 210 and trained to determine the sleep state of the user. In addition, the storage device 220 may store information processed by the processor 210, such as first biometric information and second biometric information obtained through the processor 210. In addition, the storage device 220 may store information output from the artificial intelligence model. For example, the storage device 220 may store sleep state information obtained by inputting the first biometric information and the second biometric information to the first artificial intelligence model.

According to various embodiments, the electronic device 100 may include a communication device 230. In various embodiments, the communication device 230 may support the establishment of a direct (e.g., wired) communication channel or a wireless communication channel between the electronic device 200 and an external electronic device (e.g., the sensor device 110 and the display device 120 of FIG. 1), and communication performance through the established communication channel. The communication device 230 may include one or more communication processors that operate independently from the processor 210 and support direct (e.g., wired) communication or wireless communication. According to an embodiment, the communication device 230 may include a wireless communication module (e.g., a cellular communication module, a short-range wireless communication module, or a global navigation satellite system (GNSS) communication module) or a wired communication module (e.g., a local area network (LAN) communication module, or a power line communication module). Among these communication modules, a corresponding communication module may communicate with an external electronic device through a first network (e.g., the network 104 of FIG. 1) (e.g., a short-range communication network such as Bluetooth, WiFi direct (wireless fidelity direct), or IrDA (infrared data association)), or a second network (e.g., the network 104 of FIG. 1) (e.g., a long-range communication network such as a legacy cellular network, a 5G network, a next-generation communication network, the Internet, or a computer network (e.g., the network 104 of FIG. 1) (e.g., LAN or WAN)). The various types of communication modules may be integrated into one component (e.g., a single chip), or may be implemented as a plurality of separate components (e.g., a plurality of chips).

According to various embodiments, the electronic device 100 may transmit and receive various data to and from various external devices through the communication device 230. In addition, the electronic device 100 may store the obtained data in the storage device 220. For example, the electronic device 100 may obtain biometric information of at least one user from the sensor device 110 through the communication device 230. In addition, for example, the electronic device 100 may obtain a user input related to the determination of the sleep state through the communication device 230. For example, the electronic device 100 may transmit the sleep state information to the display device 120 to display the execution screen including the sleep state information through the communication device 230.

According to various embodiments, the electronic device 100 may include a display 240. That is, the electronic device 100 may be connected to the external display device 120 to control the execution screen to be displayed, but the electronic device 100 may include the display 240. In this disclosure, the screens displayed by the display device 120 may be displayed through the display 240.

According to various embodiments, the display 240 includes a structure similar to the above-described display device 120 and may perform similar functions.

According to various embodiments, the electronic device 100 may include various components in addition to the components illustrated in FIG. 2.

According to various embodiments, the electronic device 100 may include an input module (not shown). The input module may receive commands or data to be used in the components (e.g., the processor 210) of the electronic device 100 from the outside (e.g., the user) of the electronic device 100. The input module may include, for example, a microphone, a mouse, a keyboard, a key (e.g., a button), or a digital pen (e.g., a stylus pen).

FIG. 3 illustrates a concept of controlling a function related to a sleep state determination of an electronic device.

Referring to FIG. 3, the electronic device 200 may use hardware and/or software module 300 to support functions related to health diagnosis, including sleep state determination. For example, the processor 210 may drive at least one of the data acquisition module 310, the artificial intelligence training module 320, the data processing module 330, the sleep state determination module 340, and the data output module 350 by executing instructions stored in the storage device 220. In various embodiments, a software module different from that shown in FIG. 3 may be implemented. For example, at least two modules may be integrated into one module, or one module may be divided into two or more modules. In addition, hardware and software modules can improve working performance by sharing a single function. For example, the electronic device 100 may include both an encoder implemented in hardware and an encoder implemented in software modules, and some of the data obtained through the at least one camera module may be processed in a hardware encoder and some of the others in a software encoder.

According to various embodiments, the data acquisition module 310 may include a module that provides various functions for obtaining user biometric information. In an embodiment, the data acquisition module 310 may obtain information necessary to determine the health information of the user (e.g., the subject) from an external device (e.g., the sensor device 110). For example, the data acquisition module 310 may obtain biometric information (e.g., information on the oxygen saturation of the user during the sleep or information on the electrocardiogram) for each user of at least one user from the sensor device 110. For example, the data acquisition module 310 may obtain a first set of biometric information on the oxygen saturation of each of at least one user obtained through the sensor device 120 and a second set of biometric information on the electrocardiogram of each of the at least one user.

According to various embodiments, the data acquisition module 310 may provide a UI (User Interface)/GUI (graphical UI) related to sleep state determination (sleep state inspection) to a user through the display device 120 and obtain a user input through the display device 120. For example, the user input provided through UI/GUI output through the display device of the user device 120 may be obtained through the data acquisition module 310. In this case, the UI/GUI displayed through the display device 120 may include an execution screen that guides a training request for artificial intelligence models for sleep state analysis, a re-training request for the artificial intelligence models, and/or a request for determining a user's sleep state using the artificial intelligence models.

According to various embodiments, the artificial intelligence training module 320 may include a module that provides various functions for training an artificial intelligence model to output information related to the health state of the user based on biometric information of the user. For example, the artificial intelligence training module 320 may learn an artificial intelligence model to determine a sleep state of a user by using biometric information about at least one user obtained through the data acquisition module 310.

According to various embodiments, the artificial intelligence training module 320 may train an artificial intelligence model using a first set of biometric information on the oxygen saturation of each of at least one user and a second set of biometric information on the electrocardiogram of each of at least one user obtained through the data acquisition module 310.

According to various embodiments, the artificial intelligence training module 320 may provide a function of training an artificial intelligence model in association with the data processing module 330. For example, the artificial intelligence training module 320 may train the artificial intelligence model to determine the health state of the user based on data processed through the data processing module 330.

According to various embodiments, the data processing module 330 may provide a data processing function necessary to provide a function related to the determination of the health state of the electronic device 100. For example, the data processing module 330 may convert the first biometric information set about the oxygen saturation of each of at least one user obtained through the data acquisition module 310 into a multi-dimensional (e.g., two-dimensional) to generate the first training information set. The data processing module 330 may convert the second biometric information set about the electrocardiogram of each of at least one user obtained through the data acquisition module 310 to generate the second training information set. For example, the data processing module 330 may convert the tachogram of the heart rate variability obtained based on the second biometric information set into a multi-dimensional (e.g., two-dimensional) to generate the second training information set.

According to various embodiments, the artificial intelligence training module 320 may train the artificial intelligence model using the first training information set and the second training information set processed through the data processing module 330. In this case, the artificial intelligence training module 320 may provide a function of obtaining first label data labeled the first training information set based on the sleep state classification for the user, obtaining second label data labeled the second training information set based on the sleep state classification for the user, and training the first artificial intelligence model based on the first label data and the second label data. According to various embodiments, the first artificial intelligence model trained to determine the sleep state of the user (or determine the health state) may be generated through the artificial intelligence training module 320.

According to various embodiments, the sleep state classification may refer to a classification according to the sleep state of the user. For example, the sleep state classification may include a classification according to a state in which the user breathes stably (e.g., NOMAL), and a state in which the user breathes is unstable (e.g., Apnea and/or hypopnea). For convenience of explanation, the sleep state classification has been described as two kinds, but may be classified into a plurality of states. Accordingly, the kind of label data (e.g., third label data, etc.)

According to various embodiments, the artificial intelligence training module 320 may obtain the label data through various methods. For example, the artificial intelligence training module 320 may obtain first label data labeled the first training information set according to a pre-set criterion, and obtain second label data labeled the second training information set based on the sleep state classification for the user. According to various embodiments, the artificial intelligence training module 320 may obtain label data in conjunction with the data acquisition module 310 and/or the data output module 350. For example, the display device 120 may display a training guide screen related to data train through the data output module 350, and obtain the first label data and the second label data through the data acquisition module 310. That is, the artificial intelligence training module 320 may obtain the first label data and the second label data based on the input of the user.

According to various embodiments, the sleep state determination module 340 may provide a function of determining a sleep state of a user based on biometric information of the user obtained from the data acquisition module 310. For example, the sleep state determination module 340 may determine the sleep state of the user by using first initial biometric information about the oxygen saturation of the user in sleep and second initial biometric information about the electrocardiogram obtained in conjunction with the data acquisition module 310. In this case, the sleep state determination module 340 may process and use the first initial biometric information and the second initial biometric information obtained from the data acquisition module 310 without using the same as the data processing module 330.

According to various embodiments, the sleep state determination module 340 may provide the first initial biometric information and/or the second initial biometric information to the data processing module 330 and obtain the first biometric information and/or the second biometric information.

In this case, the data processing module 330 may obtain the first biometric information obtained by converting the first initial biometric information into a multidimensional form. For example, the data processing module 330 may obtain the first biometric information obtained by converting the first initial biometric information including the oxygen saturation per time into 2D information (e.g., a 2D graph). In addition, according to various embodiments, the data processing module 330 may process the second initial biometric information to obtain the second biometric information obtained by converting the second initial biometric information into a multidimensional form. For example, the data processing module 330 may obtain the second biometric information by converting the second initial biometric information including the electrocardiogram representing the heartbeat per time into a 2D. In addition, the data processing module 330 may obtain the second biometric information by converting the second initial biometric information into a 2D. In this case, the data processing module 330 may process the second initial biometric information to convert the second initial biometric information into a 2D. For example, the data processing module 330 may obtain a plurality of peak points of the electrocardiogram and a time interval between the plurality of peak points based on the second initial biometric information, then obtain a tachogram of the heartbeat variability about the electrocardiogram based on the plurality of peak points and the time interval between the plurality of peak points, and obtain the second biometric information by converting the tachogram of the heartbeat variability into a multidimensional form.

According to various embodiments, the sleep state determination module 340 may determine the sleep state of the user by using the first biometric information and the second biometric information obtained through the data processing module 330. For example, the sleep state determination module 340 may obtain the sleep state information by inputting the first biometric information and the second biometric information to the first artificial intelligence model obtained through the artificial intelligence training module 320.

According to various embodiments, the sleep state information may be information related to a sleep disease of the user. For example, the sleep state determination module 340 may obtain sleep state information indicating whether s sleep apnea using the first artificial intelligence model. In this case, the sleep state information may also include information on the severity of sleep-related disease. For example, as a result of analyzing the first biometric information and the second biometric information, the sleep state determination module 340 may obtain sleep status information about whether it is a state without sleep apnea (e.g., about little or little occurrence of aponia and fania), whether it is a mild sleep apnea (e.g., about 5-14 times per hour of aponia or fania), whether it is a moderate sleep apnea (e.g., about 15-29 times per hour of aponia or fania), and whether it is a serious sleep apnea (e.g., about 30 times per hour or more per hour of aponia or fania). For convenience of description, the status information has been described only in the stage of the disease related to sleep apnea, but the sleep status information may include all of the diseases related to sleep, and the information about the severity of each disease may be modified according to various criteria and classifications.

According to various embodiments, the data output module 350 may include a module that provides a function of outputting various data obtained by the driving of the electronic device 100 through an output device (e.g., the display device 120). For example, the data output module 350 may control the display device 120 to output biometric information obtained from the data acquisition module 310 through the display device 120. In addition, the data output module 350 may control the display device 120 to output sleep status information obtained through the sleep status determination module 340. In addition, the data output module 350 may control the display device 120 to output a guide screen necessary for training the artificial intelligence model to determine the sleep status in association with the artificial intelligence training module 320. According to various embodiments, the data output module 350 may control various output devices (e.g., speakers) connected to the electronic device 100 to output various information. In this case, the data output module 350 may transfer various information generated through the electronic device 100 to a signal corresponding to the type of output device.

In the embodiment of FIG. 3, it can be understood that the function performed by the data acquisition module 310, the artificial intelligence training module 320, the data processing module 330, the sleep status determination module 340, and/or the data output module 350 is performed by the processor 210 executing instructions stored in the storage device 220. In addition, in various embodiments, the electronic device 100 may use one or more hardware processing circuits to perform various functions and operations disclosed in this document.

In addition, the connection relationship between the hardware/software shown in FIG. 3 is for convenience of description and does not limit the flow/direction of data or commands. The components included in the electronic device 100 may have various electrical/operative connection relationships.

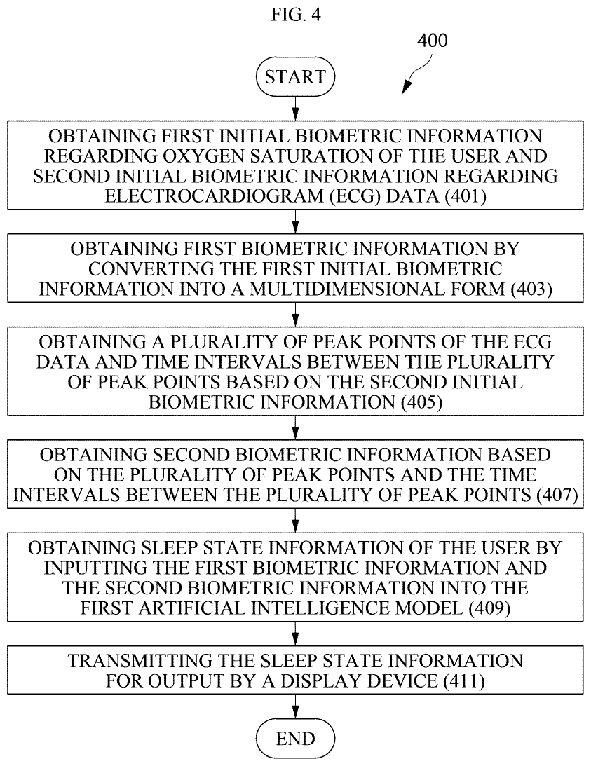

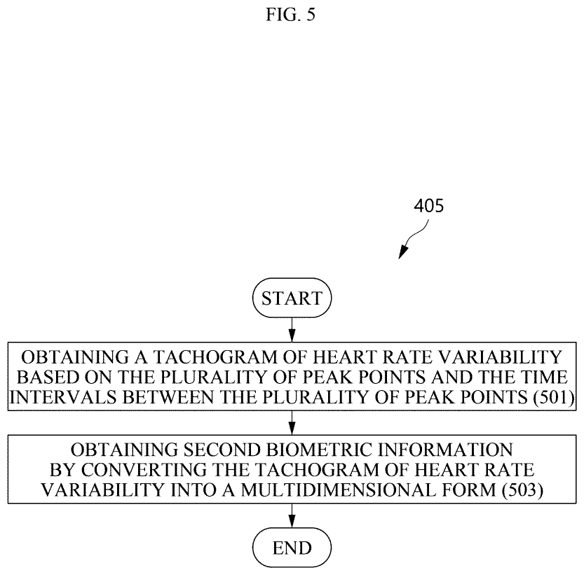

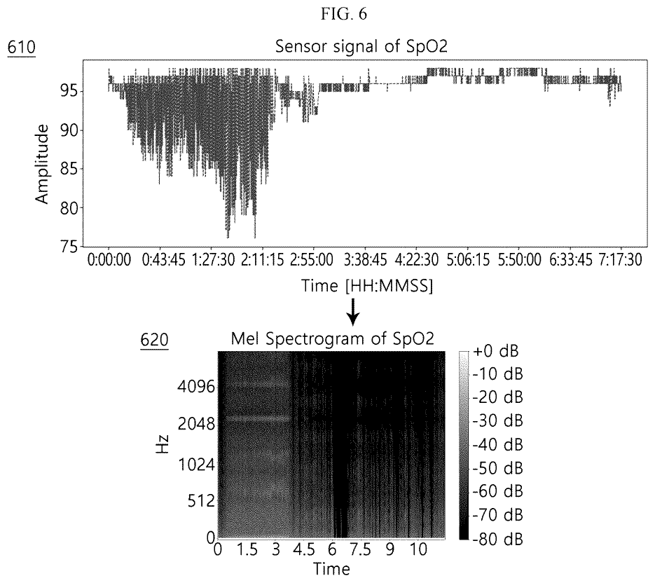

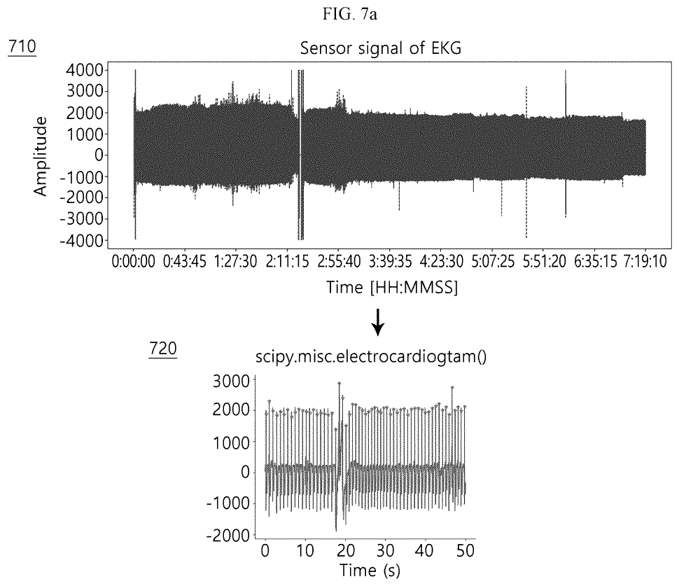

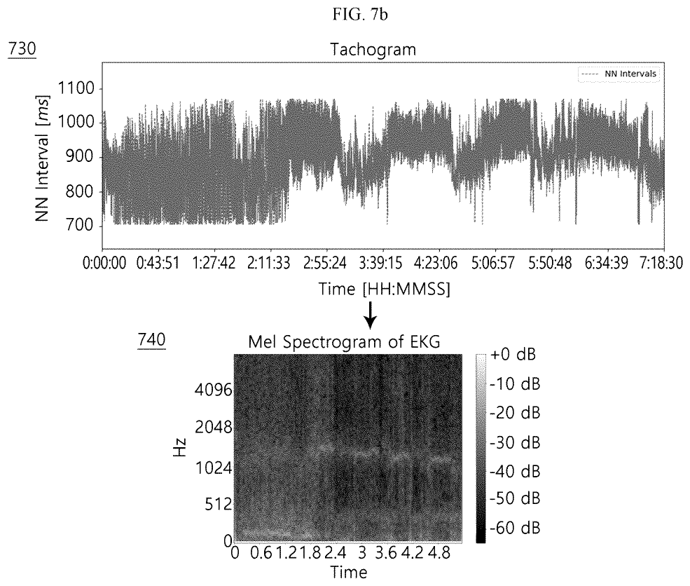

FIG. 4 is a flowchart 400 illustrating a method of determining a sleep status by an electronic device. FIG. 5 is a flowchart 500 illustrating a method of obtaining second biometric information by an electronic device. FIG. 6 is a diagram for describing obtaining first biometric information based on first initial biometric information. FIG. 7a is a diagram for describing obtaining second biometric information based on second initial biometric information. FIG. 7b is a diagram for describing obtaining second biometric information based on second initial biometric information.

The operations described below may be performed in combination with each of the operations. In addition, among the operations described below, the operation by the electronic device 100 (e.g., the electronic device 100 of FIG. 1) may mean the operation by the processor 210 of the electronic device 100.

In addition, “information” described below may be interpreted as a meaning of “data” or “signal”, and “data” may be understood as a concept that includes both analog data and digital data.

According to various embodiments, when the electronic device 100 is implemented as one device including the sensor device 110 and/or the display device 120, the operation of the sensor device 110 and/or the display device 120 may be understood as the operation of the internal configuration of the electronic device 100.

According to various embodiments, the operations illustrated in FIGS. 4 and 5 are not limited to the order illustrated, but may be performed in various orders. In addition, according to various embodiments, more operations may be performed or fewer operations may be performed than the operations illustrated in FIGS. 4 and 5.

Referring to FIG. 4, in operation 401, the electronic device 100 may obtain first initial biometric information regarding the oxygen saturation of the user and second initial biometric information regarding the electrocardiogram. For example, the electronic device 100 may obtain first initial biometric information about the oxygen saturation of the user and second initial biometric information about the electrocardiogram (ECG) through the sensor device 110 connected to the electronic device 100.

According to various embodiments, the first initial biometric information and the second initial biometric information may be represented as a one-dimensional graph.

For example, referring to FIG. 6, first initial biometric information 610 about the oxygen saturation is illustrated. The first initial biometric information 610 about the oxygen saturation may include information indicating a change in the oxygen saturation per time of the user in the sleep. For example, the first initial biometric information 610 may be represented as a graph by using the oxygen saturation of the user corresponding to the time as the vertical axis.

Referring to FIG. 7A, second initial biometric information 710 about the electrocardiogram is illustrated. The second initial biometric information 620 about the electrocardiogram may include information indicating a change in the heartbeat per time of the user in the sleep. For example, the second initial biometric information 710 may be represented as a graph (electrocardiogram) by using the heartbeat size of the user corresponding to the time as the vertical axis.

According to various embodiments, in operation 403, the electronic device 100 may obtain first biometric information obtained by converting the first initial biometric information into a multidimensional form. For example, referring to FIG. 6, the electronic device 100 may obtain first biometric information 620 by converting the first initial biometric information 610 about the oxygen saturation into a multidimensional form. Unlike various embodiments, the first biometric information may include information indicating a change in the oxygen saturation per time of the user in the sleep as the multidimensional form. For example, the first biometric information 620 may be represented as a graph by using the oxygen saturation per time of the user corresponding to the time as the horizontal axis. For example, the electronic device 100 may obtain the first biometric information 620 by transforming the first initial biometric information 610 through at least one of Fourier transform, wavelet transform, time-frequency spectrogram, and Mel-Frequency Cepstral Coefficients (MFCC).

According to various embodiments, in operation 405, the electronic device 100 may obtain a plurality of peak points of the electrocardiogram and a time interval between the plurality of peak points based on the second initial biometric information. For example, referring to FIG. 7A, information 720 about a plurality of peak points of the ECG, where the heart beat is the largest point for each time zone, may be obtained based on second initial biometric information 710 about the ECG. The electronic device 100 may obtain information 720 about a plurality of peak points for each time zone and obtain a time interval between the plurality of peak points. According to various embodiments, the information 720 about the plurality of peak points may be expressed as a graph showing the largest cardiac beat size (i.e., the peak point) for each time by using the time during the sleep of the user as the horizontal axis.

According to various embodiments, in operation 407, the electronic device 100 may obtain second biometric information based on a plurality of peak points and time intervals between the plurality of peak points. For example, the electronic device 100 may obtain second biometric information based on information 720 about a plurality of peak points.

Referring to FIG. 5, according to various embodiments, in operation 501, the electronic device 100 may obtain a tachogram of a heart rate variance based on a plurality of peak points and a time interval between the plurality of peak points. For example, referring to FIG. 7B, the electronic device 100 may obtain the tachogram 730 of the heart rate variability based on the information 720 about the plurality of peak points based on the plurality of peak points for the ECG and the time interval between the plurality of peak points. According to various embodiments, the tachogram 730 of the heart rate variability may be represented by a graph showing the time interval between the peak point and the largest cardiac beat size (i.e., the peak point) for each time, using the time during the sleep of the user as a horizontal axis. The pattern of the user's heart beat may be analyzed based on the tachogram 730 of the heart rate variance.

According to various embodiments, in operation 503, the electronic device 100 may obtain second biometric information by converting the tachogram of a heart rate variance into a multidimensional form. For example, the electronic device 100 may obtain the second biometric information 740 by converting the tachogram 730 of the heart rate variability into a multidimensional form (e.g., two-dimensional). According to various embodiments, the second biometric information may include information representing the per-hour heart rate variability (ECG) of the user in sleep in multiple dimensions (e.g., two dimensions). For example, the second initial biometric information 620 may be represented by a graph that represents the ECG-related information of the user corresponding to the time in two dimensions on the horizontal axis of the time in the sleep of the user. For example, the electronic device 100 may obtain the second biometric information 740 by transforming the heart rate variability taecogram 730 through at least one of a Fourier transform, a wavelet transform, a wavelet spectrogram, and Mel-Frequency Cepstral Coefficients (MFCC).

According to various embodiments, in operation 409, the electronic device 100 may obtain the sleep state information of the user by inputting the first biometric information and the second biometric information into the first artificial intelligence model. For example, the electronic device 100 may obtain the sleep state information of the user by inputting the first biometric information 620 and the second biometric information 740 to the first artificial intelligence model trained to determine the sleep state, described with reference to FIGS. 2 to 3.

According to various embodiments, in operation 411, the electronic device 100 may transmit the sleep state information to the display device for outputting the sleep state information. For example, the electronic device 100 may transmit the execution screen including the sleep state information to be output by the display device 120.

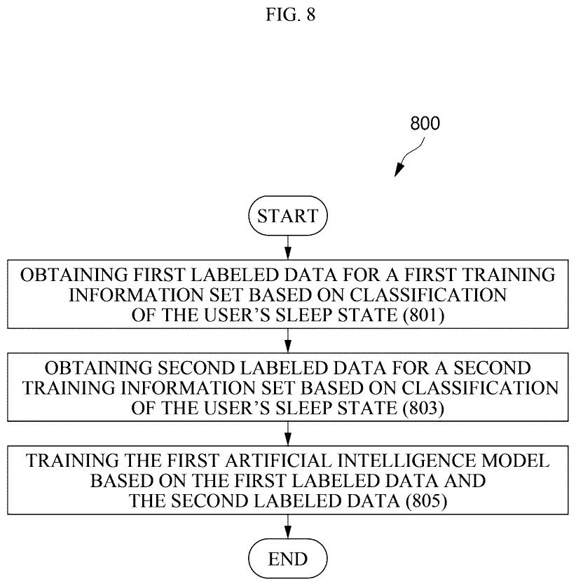

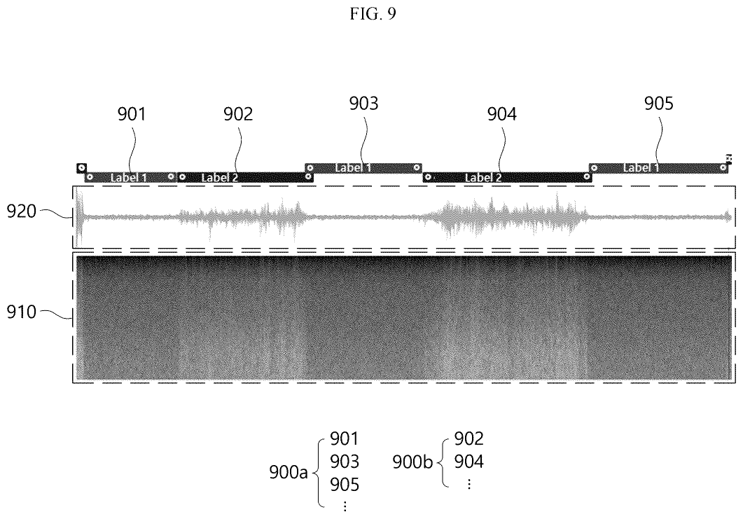

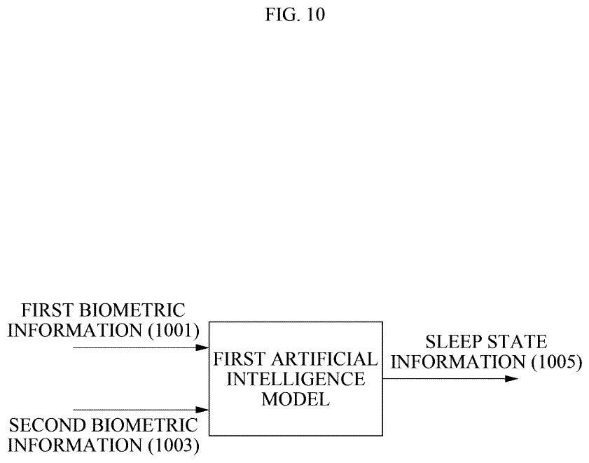

FIG. 8 is a flowchart 800 illustrating an operation in which the electronic device learns the first artificial intelligence model. FIG. 9 is a diagram illustrating that the electronic device obtains label data. FIG. 10 is a diagram illustrating that the sleep state information is obtained using the first artificial intelligence model.

Each of the operations described below may be performed in combination with each other. Also, among the operations described below, the operations by the electronic device 100 (e.g., the electronic device 100 of FIG. 1) may mean the operations by the processor 210 of the electronic device 100.

In addition, the “information” described below may be interpreted as the meaning of “data” or “signal”, and the “data” may be understood as the concept including both analog data and digital data.

According to various embodiments, the operations illustrated in FIG. 8 are not limited to the order illustrated, but may be performed in various orders. In addition, according to various embodiments, more operations may be performed or fewer operations may be performed than the operations illustrated in FIG. 8.

According to various embodiments, when the electronic device 100 is implemented as one device including the sensor device 110 and/or the display device 120, the operations for the sensor device 110 and/or the display device 120 may be understood as the operations for the internal configuration of the electronic device 100.

Referring to FIG. 8, in operation 801, the electronic device 100 may obtain first labeled data for a first training information set based on classification of the user's sleep state.

According to various embodiments, in operation 803, the electronic device 100 may obtain second labeled data for a second training information set based on classification of the user's sleep state.

According to various embodiments, the first training information set and the second training information set may be obtained based on the first biometric information set and the second biometric information set obtained through the at least one sensor device 110. For example, the electronic device 100 may store the first biometric information set about the oxygen saturation of each of at least one user obtained through the at least one sensor device 110 and the second biometric information set about the electrocardiogram of each of the at least one user in the storage device 220 (e.g., FIG. 2).

According to various embodiments, the electronic device 100 may obtain a first training information set by converting the first biometric information set into a multidimensional form, obtain a second training information set by converting a tachogram of a heart rate variability obtained based on the second biometric information set into a multidimensional form, and train the first artificial intelligence model. For example, the electronic device 100 may input the first training information set and the second training information set to the first artificial intelligence model and train the first artificial intelligence model to output sleep state information as a result value.

According to various embodiments, the electronic device 100 may train the artificial intelligence model using the first training information set and the second training information set. In this case, the electronic device 100 may obtain the first label data and the second label data through operations 801 to 803.

Referring to FIG. 9, it is illustrated that the electronic device 100 obtains the first label data based on the first training information set. Although only the acquisition of the first label data is illustrated for convenience of explanation, the electronic device 100 may obtain various label data for the second label data and other training information sets by performing similar operations. FIG. 9 illustrates labeling second initial biometric information for the oxygen saturation of one user among the biometric information sets for the oxygen saturation of at least one user included in the second biometric information set.

Referring to FIG. 9, the first state labels 900 a and the second state labels 900 b labeled for the second initial biometric information 920 and the second biometric information 910 are illustrated.

According to various embodiments, the electronic device 100 may obtain the first label data labeled the first training information set based on the sleep state classification for the user, obtain the second label data labeled the second training information set based on the sleep state classification for the user, and train the first artificial intelligence model based on the first label data and the second label data.

According to various embodiments, the sleep state classification may refer to classification according to the sleep state of the user. For example, the sleep state classification may include classification according to a state in which the user is stably breathing (e.g., NOMAL), and a state in which the user is unstable to breathing (e.g., Apnea and/or hypopnea). For convenience of explanation, the sleep state classification has been described as two kinds, but may be classified into a plurality of states. Accordingly, the type of label data (e.g., third label data or the like) may also be changed.

According to various embodiments, the electronic device 100 may label the first state labels 901, 903, and 905 for the state in which the sleep user appears to be stably breathing with respect to the second initial biometric information 920 and/or the second biometric information 910. According to various embodiments, the electronic device 100 may label the second initial biometric information 920 and/or the second biometric information 910 through the second state labels 902 and 904 for the state in which the user is unstable to breathing (e.g., Apnea and/or hypopnea).

According to various embodiments, the first state labels 900 a and the second state labels 900 b may include labels that add information or annotations to train the artificial intelligence model. According to various embodiments, the electronic device 100 may obtain the first label data and the second label data by performing similarly labeling the first training data set and the second training data set.

According to various embodiments, in operation 805, the electronic device 100 may train the first artificial intelligence model based on the first label data and the second label data.

According to various embodiments, the electronic device 100 may obtain the label data through various methods. For example, the electronic device 100 may obtain first label data obtained by labeling the first training information set according to a preset criterion, and may obtain second label data obtained by labeling the second training information set based on the sleep state classification for the user. According to various embodiments, the electronic device 100 may allow the display device 120 to display a training guide screen (e.g., a screen similar to FIG. 9) related to data training, and may obtain the first label data and the second label data through the input interface of the display device 120 and/or the electronic device 100. For example, the electronic device 100 may obtain the first label data and the second label data based on a user input.

Referring to FIG. 10, a first artificial intelligence model learned by performing the operation described with reference to FIG. 8 is illustrated. According to various embodiments, the first artificial intelligence model may receive first biometric information 1001 and second biometric information 1003 and output the sleep state information 1005.

According to various embodiments, the electronic device 100 may train the first artificial intelligence model through the operation described with reference to FIGS. 8 to 9. For example, the first artificial intelligence model may be trained to output the sleep state information of the corresponding user using the input biometric information based on the first training information set and the second training information set.

According to various embodiments, the electronic device 100 may obtain the sleep state information 1005 of the user using the first artificial intelligence model learned through the method. For example, the electronic device 100 may determine the sleep state of the user based on the sleep state information 1005 output using the first artificial intelligence model. According to various embodiments, the electronic device 100 may transmit the sleep state method to be output by the output device (e.g., the display device 120). According to various embodiments, the sleep state information may indicate information related to the sleep disorder of the user.

Accordingly, the electronic device 100 may determine whether the user has sleep disorder such as sleep apnea syndrome, obstructive sleep apnea syndrome, sleep apnea, and snoring, which are the sleep disorder of the user, to determine whether the disease related to the sleep disorder is determined. Therefore, the electronic device 100 may determine whether the user has sleep disorder based on the sleep state information.

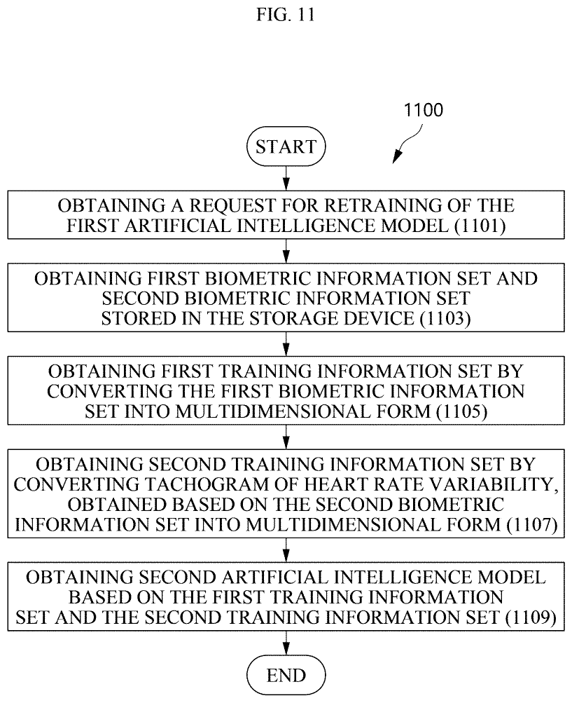

FIG. 11 is a flowchart 1100 illustrating a method of obtaining a second artificial intelligence model based on a re-training request.

Each of the operations described below may be performed in combination with each other. In addition, the operations by the electronic device 100 (e.g., the electronic device 100 of FIG. 1) may mean the operations by the processor 210 of the electronic device 100. In addition, the “information” described below may be interpreted as meaning “data” or “signal”, and “data” may be understood as concepts including both analog data and digital data.

According to various embodiments, the operations illustrated in FIG. 11 are not limited to the order and may be performed in various orders. In addition, according to various embodiments, more operations may be performed or fewer operations may be performed than the operations illustrated in FIG. 11.

According to various embodiments, when the electronic device 100 is implemented as one device including the sensor device 110 and/or the display device 120, the operation of the sensor device 110 and/or the display device 120 may be understood as the operation of the internal configuration of the electronic device 100.

Referring to FIG. 11, in operation 1101, the electronic device 100 may obtain a request for retraining the first artificial intelligence model. For example, the electronic device 100 may obtain a request for retraining the first artificial intelligence model obtained through the display device 120 using the data acquisition module 310 described with reference to FIG. 3.

According to various embodiments, in operation 1103, the electronic device 100 may obtain the first biometric information set and the second biometric information set stored in the storage device 220. For example, the electronic device 100 may obtain the first biometric information set described with reference to FIGS. 8 and 9 regarding the oxygen saturation of at least one user obtained through the sensor device 110, and obtain the second biometric information set described with reference to FIGS. 8 and 9 regarding the electrocardiogram of at least one user.

According to various embodiments, in operation 1105, the electronic device 100 may obtain a first training information set obtained by converting the first biometric information set into a multidimensional form. For example, the electronic device 100 may perform an operation similar to that described with reference to FIGS. 8 and 9 to obtain a first training information set obtained by converting the first biometric information set into a multidimensional form.

According to various embodiments, in operation 1107, the electronic device 100 may obtain a second training information set obtained by converting a tachogram of a heart rate variability based on the second biometric information set into a multidimensional form. For example, the electronic device 100 may perform an operation similar to the operation described with reference to FIGS. 8 and 9 to obtain a second training information set obtained by converting the second biometric information set into a multidimensional form.

According to various embodiments, in operation 1109, the electronic device 100 may obtain a second artificial intelligence model based on the first training information set and the second training information set. For example, the electronic device 100 may re-learn the first artificial intelligence model based on the first training information set and the second training information set to obtain a second artificial intelligence model. In various embodiments, the electronic device 100 may perform an operation similar to the operation of training the first artificial intelligence model to obtain a second artificial intelligence model as the operation of re-training the first artificial intelligence model. According to various embodiments, the electronic device 100 may obtain the second artificial intelligence model by training the artificial intelligence model set only with the initial set value.

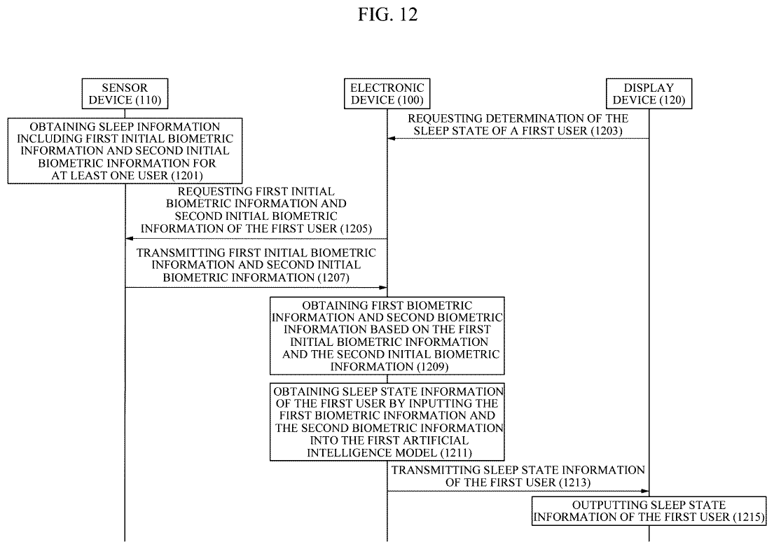

FIG. 12 is a flowchart illustrating operations of devices in a system for determining a sleep state.

Referring to FIG. 12, an electronic device 100, a sensor device 110, and/or a display device 120 are shown as devices in a system for determining a sleep state. According to various embodiments, the system for determining the sleep state may include the health state diagnosis system described with reference to FIG. 1. Therefore, descriptions similar to or overlapping with those described with reference to FIG. 1 may be omitted below.

According to various embodiments, in operation 1201, the sensor device 110 may obtain sleep information including first initial biometric information and second initial biometric information for at least one user. The sensor device 110 may store the first initial biometric information and the second initial biometric information in a storage device connected to the sensor device 110. For example, the sensor device 110 may obtain first initial biometric information on oxygen saturation obtained during sleep and second initial biometric information on electrocardiogram for at least one user, and store the first initial biometric information and the second initial biometric information.

According to various embodiments, the electronic device 100 may obtain a sleep state determination request of the first user via the display device 120, in operation 1203.

According to various embodiments, in operation 1205, the electronic device 100 may request the first initial biometric information and the second initial biometric information of the first user from the sensor device 110 in response to the sleep state determination request obtained from the display device 120.

According to various embodiments, in response to the request from the electronic device 100, in operation 1207, the sensor device 110 may transmit the first initial biometric information and the second initial biometric information to the electronic device 100. According to various embodiments, the electronic device 100 may obtain the first initial biometric information and the second initial biometric information through a communication connection with the sensor device 110, or may indirectly obtain the first initial biometric information and the second initial biometric information using an intermediate connection module device.

According to various embodiments, in operation 1209, the electronic device 100 may obtain the first biometric information and the second biometric information based on the first initial biometric information and the second initial biometric information. For example, the electronic device 100 may obtain the first biometric information obtained by converting the first initial biometric information into a multidimensional form, obtain a plurality of peak points of the electrocardiogram and a time interval between the plurality of peak points based on the second initial biometric information, and obtain the second biometric information based on the time interval between the plurality of peak points and the plurality of peak points.

Unlike various embodiments, in operation 1211, the electronic device 100 may obtain the sleep state information of the first user by inputting the first biometric information and the second biometric information to the first artificial intelligence model. For example, the electronic device 100 may obtain the sleep state information of the first user by inputting the first biometric information and the second biometric information to the first artificial intelligence model described with reference to FIGS. 2 to 3.

According to various embodiments, in operation 1213, the electronic device 100 may transmit the sleep state information of the first user to the display device 120, and the display device 120 may display a screen including the sleep state information based on a control signal of the electronic device 100. According to various embodiments, in operation 1215, the electronic device 100 may transmit various types of output devices other than the display device 120 to output the sleep state information.

According to various embodiments, the sleep state information may correspond to health state information of the user.

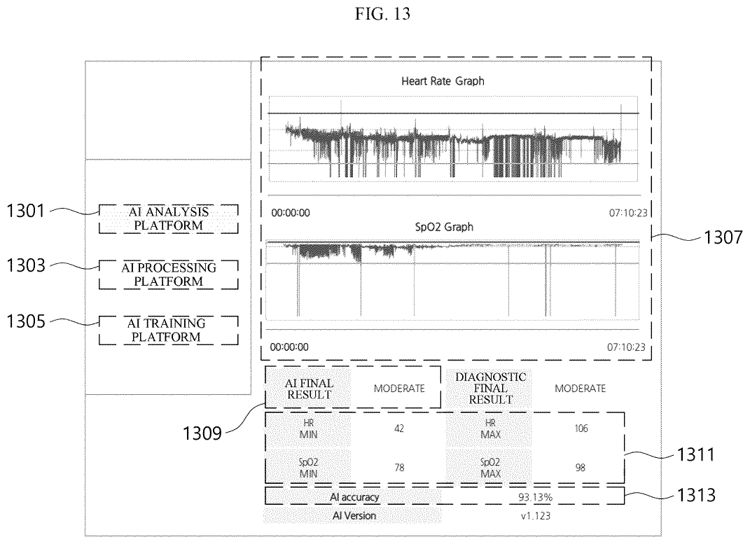

FIG. 13 illustrates a user interface output through a display device.

Referring to FIG. 13, the display device 120 may display an execution screen to provide a service related to diagnose every health state. In this case, the display device 120 may display a screen to provide a service related to health state.

According to various embodiments, the icons for the AI analysis platform 1301, the AI processing platform 1303, and the AI training platform 1305 may be displayed on the types of services related to health status diagnosis provided through the electronic device 100. According to various embodiments, when a user input for the icons 1301, 1303, and 1305 is obtained, the display device 120 may operate to activate a function related to the icon from which the user input is obtained (or to provide a function related to the icon by the electronic device).

For example, when a user input for an icon of the AI analysis platform 1301 is obtained, the display device 120 may transmit a request for health status determination (e.g., sleep status determination) for the user to the electronic device 100.

According to various embodiments, the electronic device 100 may obtain first initial biometric information and second initial biometric information, which are information on the oxygen saturation of the user and information on the electrocardiogram, based on the health state determination (e.g., sleep state determination) request, and obtain the sleep state information (or health state information) of the user by performing the operation described with reference to FIGS. 4 to 7B.

According to various embodiments, when sleep state information is obtained from the electronic device 100, the display device 120 may display an execution screen including a first visual object 1307 indicating first initial biometric information and second initial biometric information, a second visual object 1309 and 1311 for the sleep state information, and a third visual object 1313 for the accuracy of the sleep state information. For example, the display device 120 may display a graph of first initial biometric information and/or second initial biometric information, or first biometric information and/or second biometric information obtained by processing the first initial biometric information and/or the second initial biometric information, as a first visual object 1307. For example, the electronic device 120 may display information on whether the user suffers from sleep apnea through the second visual objects 1309 and 1131 regarding the sleep state information, the highest/low value of the oxygen saturation, and the highest/low value of the heartbeat, which are determined based on the obtained biometric information.

According to various embodiments, when a user input for the AI processing platform 1303 is obtained through the display device 120, a re-training operation for the artificial intelligence model described with reference to FIGS. 3 and 11 may be performed through the electronic device 100. Accordingly, the display device 120 may display an execution screen related to the re-training operation for the first artificial intelligence model.

According to various embodiments, when a user input for the AI training platform 1305 is obtained through the display device 120, the training operation for the artificial intelligence model described with reference to FIGS. 3 and 8 to 10 may be performed through the electronic device 100. Accordingly, the display device 120 may display an execution screen related to the re-training operation for the first artificial intelligence model.

As described above, according to various embodiments, an electronic device may include a communication device, a storage device storing a first artificial intelligence model trained to determine a sleep state, and at least one processor, wherein the at least one processor may be configured to obtain first initial biometric information about oxygen saturation of a user in sleep and second initial biometric information about electrocardiogram obtained through at least one sensor device, obtain first biometric information obtained by converting the first initial biometric information into a multidimensional form, obtain a plurality of peak points of the electrocardiogram and a time interval between the plurality of peak points based on the second initial biometric information, obtain second biometric information based on the plurality of peak points and the time interval between the plurality of peak points, obtain sleep state information of the user by inputting the first biometric information and the second biometric information to the first artificial intelligence model, and transmit the sleep state information to a display device connected to the electronic device through the communication device.

According to various embodiments, the at least one processor may be configured to obtain a tachogram of a heart rate variation about the electrocardiogram based on the plurality of peak points and the time interval between the plurality of peak points, and obtain second biometric information by converting the tachogram of the heart rate variation into a multidimensional form.

According to various embodiments, the sleep state information may be information related to a sleep disease, and the sleep disease may include at least one of sleep apnea syndrome, obstructive sleep apnea syndrome, sleep apnea, and snoring.

According to various embodiments, the at least one processor may be configured to control the storage device to store a first biometric information set about oxygen saturation of each of at least one user obtained through the at least one sensor device and a second biometric information set about electrocardiogram of each of the at least one user.

According to various embodiments, the at least one processor may be configured to obtain a first training information set obtained by converting the first biometric information set into a multidimensional form, and obtain a second training information set obtained by converting the tachogram of the heart rate variation based on the second biometric information set into a multidimensional form, and the first artificial intelligence model may be a model trained based on the first training information set and the second training information set.

According to various embodiments, the at least one processor may be configured to obtain first label data obtained by labeling the first training information set based on a sleep state classification for a user, obtain second label data obtained by labeling the second training information set based on the sleep state classification for a user, and train the first artificial intelligence model based on the first label data and the second label data.

According to various embodiments, the at least one processor may be configured to control the display device to display a training guide screen related to obtain the first label data and the second label data, and obtain the first label data and the second label data based on an input obtained in relation to the training guide screen through the display device.

According to various embodiments, the at least one processor may be configured to obtain a re-training request of the first artificial intelligence model, obtain the first biometric information set and the second biometric information set stored in the storage device based on the acquisition of the re-training request, obtain the first training information set obtained by converting the first biometric information set into a multidimensional form, obtain the second training information set obtained by converting a tachogram of a heart rate variability obtained based on the second biometric information set into a multidimensional form, and obtain a second artificial intelligence model based on the first training information set and the second training information set.

According to various embodiments, the at least one sensor device may include at least one of an optical fiber oxygen sensor, a membrane oxygen sensor, an ECG sensor, an EKG sensor, a PPG sensor, a biometric impedance sensor, and an ultrasonic sensor.

As described above, the operation method of the electronic device may include the operation of obtaining first initial biometric information on the oxygen saturation of the user in sleep and second initial biometric information on the electrocardiogram obtained through the at least one sensor device, the operation of obtaining first biometric information obtained by converting the first initial biometric information into a multidimensional form, the operation of obtaining a time interval between a plurality of peak points of the electrocardiogram and the plurality of peak points based on the second initial biometric information, the operation of obtaining second biometric information based on the time interval between the plurality of peak points and the plurality of peak points, the operation of obtaining sleep state information of the user by inputting the first biometric information and the second biometric information to the first artificial intelligence model, and the first artificial intelligence model may be a model trained to determine the sleep state and may be transmitted to a display device connected to the electronic device to output the sleep state information.

According to various embodiments, the operation method of the electronic device may further include the operation of obtaining a tachogram of a heart rate variability based on the plurality of peak points and the time interval between the plurality of peak points, and the operation of obtaining the second biometric information by converting the tachogram of the heart rate variability into a multidimensional form.

According to various embodiments, the sleep state information may be information related to sleep disease, and the sleep disease may include at least one of sleep apnea syndrome, obstructive sleep apnea syndrome, sleep apnea, and snoring.

According to various embodiments, the operation method of the electronic device may further include the operation of controlling the storage device connected to the electronic device to store the first biometric information set and the second biometric information set related to the electrocardiogram of each of at least one user obtained through the at least one sensor device.

According to various embodiments, the operation method of the electronic device may further include the operation of obtaining the first training information set obtained by converting the first biometric information set into a multidimensional form, and the operation of obtaining the second training information set obtained by converting the tachogram of the heart rate variability based on the second biometric information set, and the first a model trained based on the first training information set and the second training information set.

According to various embodiments, the operating method of the electronic device may further include obtaining first label data labeled with the first training information set based on the sleep state classification for the user, obtaining second label data labeled with the second training information set based on the sleep state classification for the user, and training the first artificial intelligence model based on the first label data and the second label data.

According to various embodiments, the operating method of the electronic device may further include controlling the display device to display the training guide screen related to the acquisition of the first label data and the second label data, and obtaining the first label data and the second label data based on an input obtained in relation to the training guide screen through the display device.

According to various embodiments, the operating method of the electronic device may further include obtaining a re-training request of the first artificial intelligence model, obtaining the first biometric information set and the second biometric information set from a storage device connected to the electronic device based on the acquisition of the re-training request, obtaining the first training information set obtained by converting the first biometric information set into a multidimensional form, obtaining the second training information set obtained by converting a tachogram of a degree of heart variation obtained based on the second biometric information set into a multidimensional form, and obtaining the second artificial intelligence model trained based on the first training information set and the second training information set.

According to various embodiments, the at least one sensor device may include at least one of an optical fiber oxygen sensor, a membrane oxygen sensor, an ECG sensor, an EKG sensor, a PPG sensor, a biometric impedance sensor, and an ultrasonic sensor.

As described above, the health status diagnosis system includes at least one sensor device that obtains biometric information including oxygen saturation and ECG of the user for a certain period of time, a display device, and a server that stores the first artificial intelligence model trained to determine the sleep state, wherein the server may obtain first initial biometric information about the oxygen saturation of the user and second initial biometric information about the ECG obtained through the at least one sensor device, obtain first biometric information obtained by converting the first initial biometric information into a multidimensional form, obtain a time interval between a plurality of peak points of the ECG and the plurality of peak points based on the second initial biometric information, obtain second biometric information based on the time interval between the plurality of peak points and the plurality of peak points, input the first biometric information and the second biometric information to the first artificial intelligence model to obtain the health status information of the user, and transmit the health status information to the display device, and the display device may display a screen including the health status information.

In this disclosure, each of phrases such as “a or b”, “a or b”, “a, b, or c”, “a, b, or c”, and “a, b, may include any one of the items listed together in the corresponding phrases, or all combinations thereof.

The terms “first”, “second”, or “first” or “second” may simply be used to distinguish a corresponding component from another corresponding component, and do not limit the components in other aspects (e.g., important or order).

The term “module” used in various embodiments of the present disclosure may include a unit implemented in hardware, software, or firmware. For example, the term “logic”, “logical block”, “component”, or “circuit” may be used interchangeably. The module may be an integrated component or a minimum unit of the component or a part thereof that performs one or more functions.

Various embodiments of the present disclosure may be implemented as software (e.g., a program) including one or more instructions stored in a memory (e.g., an internal memory or an external memory) that can be read by a device (e.g., the electronic device 100). The memory may be represented as a storage medium.

According to an embodiment, the method according to various embodiments disclosed herein may be included and provided in a computer program product. The computer program product may be traded between a seller and a buyer as a product. The computer program product may be distributed in the form of a storage medium (e.g., a compact disc read only memory (CD-ROM)) that can be read by a device, or distributed (e.g., downloaded or uploaded) online, directly, through an application store or between two user devices.

According to various embodiments, each component (e.g., module or program) of the above-described components may include a single entity or a plurality of entities, and some of the plurality of entities may be separately disposed in other components. According to various embodiments, one or more components or operations among the above-described corresponding components may be omitted, or one or more other components or operations may be added. Additionally or alternatively, a plurality of components (e.g., modules or programs) may be integrated into one component. In this case, the integrated component may perform one or more functions of each component of the plurality of components in the same or similar manner as that performed by the corresponding component among the plurality of components before the integration.

According to various embodiments, operations performed by a module, a program, or another component may be executed sequentially, in parallel, repeatedly, or heuristically, one or more of the operations may be executed in a different order, omitted, or one or more other operations may be added.

1. An electronic device comprising:
a communication device;
a storage device storing a first artificial intelligence model trained to determine a sleep state; and
at least one processor,
wherein the at least one processor is configured to:
obtain first initial biometric information regarding oxygen saturation of a user in sleep state and second initial biometric information regarding electrocardiogram (ECG) data via at least one sensor device;
obtain first biometric information by converting the first initial biometric information into a multidimensional form;
obtain a plurality of peak points of the ECG data and time intervals between the plurality of peak points, based on the second initial biometric information;
obtain second biometric information based on the plurality of peak points and the time intervals between the plurality of peak points;
input the first biometric information and the second biometric information into the first artificial intelligence model to obtain sleep state information of the user; and
transmit the sleep state information to a display device connected to the electronic device via the communication device for output the sleep state information.

2. The electronic device of claim 1, wherein the at least one processor is configured to:
obtain a tachogram of heart rate variability based on the plurality of peak points and the time intervals between the plurality of peak points; and
obtain the second biometric information by converting the tachogram of heart rate variability into a multidimensional form.

3. The electronic device of claim 1, wherein the sleep state information is information related to a sleep disorder, and
wherein the sleep disorder includes at least one of sleep apnea syndrome, obstructive sleep apnea syndrome, sleep apnea, and snoring.

4. The electronic device of claim 1, wherein the at least one processor is configured to control the storage device to store a first biometric information set regarding oxygen saturation and a second biometric information set regarding ECG data for each of at least one user obtained via the at least one sensor device.

5. The electronic device of claim 4, wherein the at least one processor is configured to:
obtain a first training information set by converting the first biometric information set into a multidimensional form;
obtain a second training information set by converting a tachogram of heart rate variability, obtained based on the second biometric information set, into a multidimensional form; and
wherein the first artificial intelligence model is trained based on the first training information set and the second training information set.

6. The electronic device of claim 5, wherein the at least one processor is configured to:
obtain first labeled data by labeling the first training information set based on classification of the sleep state of the user;
obtain second labeled data by labeling the second training information set based on the classification of the sleep state of the user; and
train the first artificial intelligence model based on the first labeled data and the second labeled data.

7. The electronic device of claim 6, wherein the at least one processor is configured to:
control the display device to display a training guide screen related to obtaining the first labeled data and the second labeled data; and
obtain the first labeled data and the second labeled data based on an input obtained through the training guide screen via the display device.

8. The electronic device of claim 5, wherein the at least one processor is configured to:
obtain a retraining request for the first artificial intelligence model;
obtain the first biometric information set and the second biometric information set stored in the storage device based on obtaining the retraining request;
obtain the first training information set by converting the first biometric information set into a multidimensional form;
obtain the second training information set by converting a tachogram of heart rate variability, obtained based on the second biometric information set, into a multidimensional form; and
obtain a second artificial intelligence model trained based on the first training information set and the second training information set.

9. The electronic device of claim 1, wherein the at least one sensor device includes at least one of a fiber optic oxygen sensor, membrane oxygen sensor, ECG sensor, EKG sensor, PPG sensor, bio-impedance sensor, and ultrasound sensor.

10. A method of operating an electronic device, comprising:
obtaining first initial biometric information regarding oxygen saturation and second initial biometric information regarding electrocardiogram (ECG) data of a sleeping user via at least one sensor device;
obtaining first biometric information by converting the first initial biometric information into a multidimensional form;
obtaining a plurality of peak points of the ECG data and time intervals between the plurality of peak points based on the second initial biometric information;
obtaining second biometric information based on the plurality of peak points and the time intervals between the plurality of peak points;
inputting the first biometric information and the second biometric information into a first artificial intelligence model trained to determine a sleep state to obtain sleep state information of the user; and
transmitting the sleep state information to a display device connected to the electronic device for output the sleep state information.

11. The method of claim 10, further comprising:
obtaining a tachogram of heart rate variability based on the plurality of peak points and the time intervals between the plurality of peak points; and
obtaining the second biometric information by converting the tachogram of heart rate variability into a multidimensional form.

12. The method of claim 10, wherein the sleep state information is information related to a sleep disorder, and the sleep disorder includes at least one of sleep apnea syndrome, obstructive sleep apnea syndrome, sleep apnea, and snoring.

13. The method of claim 10, further comprising controlling a storage device connected to the electronic device to store a first biometric information set regarding oxygen saturation and a second biometric information set regarding ECG data for each of at least one user obtained through the at least one sensor device.

14. The method of claim 13, further comprising:
obtaining a first training information set by converting the first biometric information set into a multidimensional form;
obtaining a second training information set by converting a tachogram of heart rate variability, obtained based on the second biometric information set, into a multidimensional form;
wherein the first artificial intelligence model is trained based on the first training information set and the second training information set.

15. The method of claim 14, further comprising:
obtaining first labeled data by labeling the first training information set based on classification of the sleep state of the user;
obtaining second labeled data by labeling the second training information set based on the classification of the sleep state of the user; and
training the first artificial intelligence model based on the first labeled data and the second labeled data.

16. The method of operation of the electronic device according to claim 15, further comprising:
controlling the display device to display a training guide screen related to the acquisition of the first labeled data and the second labeled data; and
obtaining the first labeled data and the second labeled data based on an input received via the display device in association with the training guide screen.

17. The method of operation of the electronic device according to claim 14, further comprising:
obtaining a request for retraining of the first artificial intelligence model;
obtaining, based on the retraining request, the first biometric information set and the second biometric information set from a storage device connected to the electronic device;
obtaining the first training information set by converting the first biometric information set into a multidimensional form;
obtaining the second training information set by converting a tachogram of heart rate variability, obtained based on the second biometric information set, into a multidimensional form; and
obtaining a second artificial intelligence model trained based on the first training information set and the second training information set.

18. The method of operation of the electronic device according to claim 10, wherein the at least one sensor device includes at least one of a fiber optic oxygen sensor, a membrane oxygen sensor, an ECG sensor, an EKG sensor, a PPG sensor, a bio-impedance sensor, or an ultrasound sensor.

19. A health condition diagnosis system comprising:
at least one sensor device configured to obtain biometric information, including oxygen saturation and electrocardiogram (ECG) data of a user, over a period of time;
a display device; and
a server storing a first artificial intelligence model trained to determine a sleep state,
wherein the server is configured to:
obtain, via the at least one sensor device, first initial biometric information regarding the user's oxygen saturation and second initial biometric information regarding the user's electrocardiogram (ECG) data;
obtain first biometric information by converting the first initial biometric information into a multidimensional form;
obtain a plurality of peak points of the ECG data and time intervals between the plurality of peak points based on the second initial biometric information;
obtain second biometric information based on the plurality of peak points and the time intervals between the plurality of peak points;
input the first biometric information and the second biometric information into the first artificial intelligence model to obtain health condition information of the user;
transmit the health condition information to the display device; and
wherein the display device is configured to display a screen including the health condition information.
